(19) United States
(12) Patent Application Publication
(10) Pub. No.: US 2025/0381200 A1
(43) Pub. Date: Dec. 18, 2025

(54) SINGLE-INJECTION METHODS AND FORMULATIONS TO CONTROL THE ONSET OF ESTRUS AND OVULATION IN BOVINE, CAPRINE, OVINE, CAMELID AND OTHER FEMALE ANIMALS

(71) Applicant: Therio, LLC, Manhattan, KS (US)

(72) Inventor: John L. CURTIS, Manhattan, KS (US)

(21) Appl. No.: 19/298,553

(22) Filed: Aug. 13, 2025

Related U.S. Application Data
(60) Provisional application No. 63/660,978, filed on Jun. 17, 2024.

(51) Int. Cl. A61K 31/57; A61K 9/00; A61K 9/16; A61K 38/24; A61P 15/08

(52) U.S. Cl. CPC A61K 31/57; A61K 9/0019; A61K 9/1647; A61K 38/24; A61P 15/08

(57) ABSTRACT

The present invention relates to assisted reproductive management technologies common to animal husbandry, more specifically technologies and processes enabling timed artificial insemination (TAI) and timed embryo transfer (TET), superovulation for multiple ovulation embryo transfer (MOET) and superstimulation for ovum pickup (OPU) prior to in-vitro fertilization. More specifically, the present invention is directed to simplified, single-injection methods and formulations to control the onset of estrus and ovulation occurring within the reproductive technologies described above. The formulations focus on progesterone (P4) and luteinizing hormone (LH).

### CROSS REFERENCE TO RELATED APPLICATION

The present invention claims the benefit of priority of U.S. Provisional Patent Application No. 63/660,978 filed Jun. 17, 2024. The contents of this application are incorporated by reference in their entirety.

The present application is in conjunction with U.S. patent application No. 17,828,412 filed May 31, 2022, which is a divisional of U.S. patent application Ser. No. 15/912,101, filed Mar. 5, 2018, which claims the benefit of priority date of U.S. Provisional Patent Application Ser. No. 62/527,084, filed Jun. 30, 2017, entitled SIMPLIFIED, SINGLE-INJECTION METHOD TO INDUCE AND CONTROL THE SYNCHRONOUS GROWTH AND OVULATION OF MULTIPLE OVARIAN FOLLICLES (SUPEROVULATION) IN BOVINE, OVINE, CAMELID AND OTHER FEMALE ANIMALS. The contents of these applications is herein incorporated by reference in their entirety.

### FIELD OF THE INVENTION

The present invention relates to assisted reproductive management technologies common to animal husbandry, more specifically technologies and processes enabling timed artificial insemination (TAI) and timed embryo transfer (TET), superovulation for multiple ovulation embryo transfer (MOET) and superstimulation for ovum pickup (OPU) prior to in-vitro fertilization. More specifically, the present invention is directed to simplified, single-injection methods and formulations to control the onset of estrus and ovulation.

### BACKGROUND OF THE INVENTION

One of the reproductive management processes within animal husbandry is commonly referred to as embryo transfer (ET) technology which is comprised of the two sub-disciplines of ovum pickup (OPU) followed by in-vitro fertilization (IVF); and multiple ovulation embryo transfer (MOET) followed by artificial insemination. Both sub-disciplines deliver the same outcome of multiple embryos which are then transferred immediately into uteri of surrogate hosts, or frozen for transfer later. In both cases the estrous cycle of the surrogate embryo hosts (i.e. recipients) is managed to control (a) the length of the estrous cycle, (b) when the dominant follicle ovulates and (c) when estrus occurs. The process of timed artificial insemination (AI) is similarly managed to control (a) the length of the estrous cycle, (b) when the dominant follicle ovulates and (c) when estrus occurs.

While AI has been practiced commercially the past 80 years and MOET the past 40 years, commercial acceptance of IVF produced embryos began approximately 10 years ago.

Existing methods and systems for IVF and MOET include: EP0021234B1, EP0298990B1, U.S. Pat. Nos. 3,499,445, 3,835,108, 3,860,701, 4,005,063, 4,008,209, 4,159,980, 4,670,419, 4,762,717, 4,780,451, 4,975,280, 5,162,306, 5,512,303, 5,589,457, 5,633,014, 5,650,173, 5,747,058, 5,941,844, 6,028,057, 6,573,254, 7,151,083, 7,205,281, 7,446,090, 7,740,884, 7,741,268, 8,530,419, 8,905,913, 8,927,496, 8,937,044, 9,018,165, 9,351,818, 9,352,011, US 20050130894, US 20060264372, 20070173450, US 20070197435, US 20090036384, US 20120046519, US 20130041210, US 20130085321, CN101129333A, CN104800834A, U.S. Pat. Nos. 4,599,227, 7,563,763, 7,629,113, 8,518,881, US 20080312151, US 20140335193, US 20150335713, US 20160250333, WO199516459A1.

Prior to OPU the oocyte donor's estrous cycle and dominant follicle presence is managed. Prior to and during superovulation the embryo donor's estrous cycle, dominant follicle and onset of estrus are managed. Prior to timed artificial insemination (TAI) and timed embryo transfer (TET) the animal's estrous cycle, dominant follicle and onset of estrus are managed.

The method of superovulation which has been the gold standard protocol the past 40 years due to its predictable embryo yield is to administer eight injections of follicle stimulating hormone (FSH) at half-day intervals. Prior to and throughout FSH treatment, a progesterone (P4) impregnated device (CIDR® in the US market) is placed vaginally which maintains high, sustained systemic P4. A prostaglandin such as Lutalyse (dinoprost) or Estrumate (D-cloprostenol) is injected 72 to 96 hours after initiation of treatment along with the eighth FSH injection. In addition to the eight FSH injections, in the absence of an approved injectable LH, an injection of gonadotrophin releasing hormone (GnRH) is normally administered two days before the start of FSH which serves to stimulate the release of endogenous LH, which causes rupture (removal) of the dominant ovarian follicle thereby prompting a new wave of follicular growth to begin. A second GnRH injection is routinely administered in MOET programs two days after the final FSH injection to stimulate the release of endogenous LH to control and predict the donor's ovulation period. This second GnRH injection is administered with the hope and desire that it will manipulate, stimulate and control the onset of the donor's endogenous LH surge culminating in synchronous ovulation over a desired period of time resulting in a high fertilization rate. Endogenous GnRH originates in the hypothalamus and travels systemically to the pituitary gland with the expectation that as a releasing hormone, it will travel systemically to the pituitary and stimulate the critical pulsatile releases of LH (the LH surge) from the pituitary which then travels systemically to the ovaries to induce synchronous and complete ovulation over a time period. Unfortunately, the addition of exogenous GnRH during a MOET procedure does not always result in synchronous nor complete ovulation of all matured follicles, both of which are required to achieve a high fertilization rate and high embryo yield. Physiologically LH is the hormone directly responsible for the induction of synchronous and complete ovulation, not GnRH. Historically as well as currently, the popular GnRH-FSH-GnRH MOET protocol requires injecting the animal at minimum 10 different times over eight-nine days.

There is no FDA approved veterinary-use LH in the US. For that reason, current, popular superovulation protocols in the US inject GnRH prior to the start of FSH injections to stimulate the endogenous release of LH to cause removal of the dominant follicle. GnRH is injected once again 8-9 days later (after FSH has stimulated growth and maturity of follicles) to initiate the chain of events leading to ovulation. Administering exogenous GnRH does not explicitly achieve the end-goal of ovulation, and physiologically GnRH is not directly responsible for causing ovulation, versus administering LH which is the hormone directly responsible for ovulation.

Disadvantageously however, today's gold-standard superovulation protocol is rather inefficient overall due to all known inducing agents having a very short biological half-life of 0.3-5.0 hours which mandates repeated dosing at specific timed intervals throughout a donor's superovulation or superstimulation period. The protocol is inefficient due to repeated dosing requirements (10 injections for MOET, 4-6 for OPU) of donor animals with stimulating and/or releasing/inducing agents on prescribed days at 12-hour intervals over 8-9 days. The protocol can be challenging for the veterinarian or cattle/animal owner to comply with. The current protocol is (a) difficult to achieve 100% compliance because it requires strict attention to multiple details and personnel over 8-9 days at locations normally distant from the MOET or OPU technician; (b) it is time and labor intensive (therefore costly) requiring dedicated on-farm or in-clinic personnel on at least ten occasions at defined times and days to identify, gather, confine, physically restrict to insert the CIDR® and lastly inject the MOET or IVF donor(s), embryo recipients or animals synchronized for insemination; (c) the current protocol subjects donors, semen and embryo recipients and personnel to stress and potential physical injury; and (d) the increased donor stress associated with the gathering/handling and injection sequence has been shown to negatively affect the donor's superovulatory response, fertilization rate and milk production. The entire sequence of manual insertion of a CIDR® progesterone device (PD) into donor's vagina, initial GnRH injection followed by twice daily FSH injections, concluding with removal of the PD and a final GnRH injection is stressful to the animals being cycled for AI, for timed ET, for embryo or oocyte donor animals and subjects personnel to increasing exposure for physical injury with each sequential requirement to gather, confine, restrain and inject animals often weighing 1500 pounds or more.

The objective of designing and achieving equivalent outcomes from reduced handling days and less physical risk exposure is very desirable.

A common practice when synchronizing groups of cattle for performing timed artificial insemination or timed embryo transfer over the last few decades has been to “group synchronize” by manually placing (by hand, in stall) a progesterone impregnated device (“PD”) e.g. CIDR® into the vagina of a cow or heifer or other female, two days later injecting with GnRH to remove the dominant follicle, then seven days after CIDR insertion manually removing (by hand, in stall) the PD and dosing with a prostaglandin hormone via injection. Two days after PD removal and hormone injection, the animal is manually (by hand, in stall) injected with GnRH to induce release of LH to cause estrus followed by ovulation. Typically, if desired, the animal is inseminated 6-18 hours after estrus or is implanted with an embryo 7 days after estrus. The synchronization process described above requires sorting and bringing the animal on four or five different days into the handling stall to receive a procedure/treatment, as outlined below.
1^{st} day cattle sorting and handling event: insert CIDR which inhibits estrus by suppressing estrogen;
2^{nd} day cattle sorting and handling event: inject with GnRH to remove dominant follicle;
3^{rd} day cattle sorting and handling event: remove CIDR & inject w/prostaglandin;
4^{th} day cattle sorting and handling event: inject with GnRH; then inseminate;
5^{th} day cattle sorting and handling event: if not inseminating, wait 7 days then transfer an embryo into the cow.

### Designing and Achieving Equivalent Outcomes From Reduced Handling Days is Very Desirable

A very popular protocol for synchronizing the estrous cycle of cattle prior to being implanted with an embryo produced by OPU/IVF or MOET, or prior to being artificially inseminated is known as the “7 &7 Synch: An Estrus Synchronization Protocol for Postpartum Beef Cows”.

Researchers at the University of Missouri recently evaluated a new protocol for synchronization of estrus among postpartum beef cows. This protocol was found to be highly effective both for cows receiving embryo transfer (ET) and cows receiving fixed-time artificial insemination (AI). Extensive field trials with the 7 & 7 Synch observed improvements in the proportion of cows expressing estrus and in the proportion of cows becoming pregnant to embryo transfer or to AI (see https://extension.missouri.edu/publications/g2023 and enclosed in its entirety by reference).

Such known and described protocols such as the 7 &7 Synch when used in superstimulation, superovulation and timed AI or timed ET protocols designed to suppress estrus and direct the subsequent onset of estrus disadvantageously involve multiple steps requiring precise timing and the herding of animals into ranching facility stalls on four or five different days to perform repeated semi-invasive veterinary procedures which are stressful to the donor and recipient animals, and subjects personnel and animals to the increasing exposure for physical injury with each sequential requirement to gather, confine, restrain and inject animals often weighing 1500 pounds or more.

Designing and achieving equivalent outcomes from reduced handling days is highly desirable.

Thus, it is desired to provide method and formulations that solve the disadvantages in the prior art for controlling the estrous cycle, dominant follicle removal and onset of estrus during superstimulation and superovulation protocols, and during timed embryo transfer and insemination protocols.

It is an objective of the present invention to reduce the total cattle sorting and handling event days when synchronizing cattle cycles for TAI and TET from 4 days to 2 days; and from 3 days to 2 days when synchronizing donors for OPU or MOET.

Accordingly, it is desirable to improve upon the disadvantages of the current prior art methods to achieve equivalent outcomes from reducing handling days.

### SUMMARY OF THE INVENTION

It is an object of the present invention to provide methods and formulations that solve the disadvantages in the prior art for controlling the estrous cycle during superstimulation and superovulation protocols, and during timed embryo transfer and insemination protocols. In addition, it is an object of the present invention to provide a method and system that solves the disadvantages in the prior art protocol for caprine, ovine, camelid and other female animals.

It is an object of the present invention to provide a method and formulation for a simplified, single-injection method to control the duration of the estrous cycle, control dominant follicle removal and control when estrus will occur in bovine, caprine, ovine, camelid and other female animals enabling groups of cattle on the same day to be inseminated or to receive an embryo; or to enable the subsequent collection of (a) multiple oocytes if conducting via in-vitro fertilization, or (b) multiple embryos if conducting via multiple ovulation embryo transfer.

It is an object of the present invention to provide a sustained, controlled release method and formulation that involves administering a drug formulation in a single administration for inducing and controlling the duration of the estrous cycle, control dominant follicle removal and thereby control when estrus will occur in bovine, ovine, caprine, camelid and other female animals when subjected to protocols for ovum pickup/superstimulation, superovulation, timed embryo transfer and times artificial insemination

It is an object of the present invention to provide a drug formulation comprising a dual-hormone microsphere matrix of animal or recombinant technology origin, encapsulated by an engineered controlled release agent.

It is an object of the present invention to provide an injectable drug formulation that suppresses estrus via progesterone (P4) and effects dominant follicle removal via LH.

It is an object of the present invention to provide a drug formulation that controls the magnitude and duration of P4 in the blood stream via engineered controlled release technology agents.

It is an object of the present invention to provide a drug formulation that controls the timing and removal of the dominant follicle(s) and suppression of estrus via an engineered controlled release technology.

It is an object of the present invention to provide systems and methods for administering a hormone microsphere matrix to an animal, the microsphere matrix including controlled release agents, wherein the step of administering the microsphere matrix to the bovine animal is performed via a single administration, and wherein the hormone microsphere matrix suppresses estrus and induces dominant follicle removal when used prior to OPU for IVF, during superstimulation when used in the animal for MOET, and when used in the animal to synchronize estrus for timed AI or timed ET.

It is an object of the present invention for engineered controlled release LH in one formulation to cause dominant follicle removal (DFR) immediately upon injection; in other formulations the engineered controlled release LH causes dominant follicle removal (DFR) two days after injection or 7 days after injection.

It is an object of the present invention to provide methods and systems that include all of the above advantages and combinations of all the above objects and advantages.

These and other objects of the invention are achieved by providing a method of suppressing estrus and effecting DFR in bovine animals comprising the steps of administering a hormone microsphere matrix having a diameter ranging from 50-70 microns to an animal, the hormone microsphere matrix including a controlled release agent, wherein the step of administering the microsphere matrix to the animal is performed via a single administration, and wherein the microsphere matrix induces estrus suppression over a defined period of time, and causes DFR during a defined window of time.

In certain embodiments, the hormone microsphere matrix includes an active pharmaceutical ingredient (API).

In certain embodiments, the API is P4 and/or LH.

In certain embodiments, the hormone microsphere matrix comprises at least one polymer.

In certain embodiments, the hormone microsphere matrix comprises polylactide (PLA) or polylatic co-glycolic acid (PGLA).

In certain embodiments, the hormone microsphere matrix comprises an organic polymer.

In certain embodiments, at least one polymer includes poly (dl-lactide), lactide/glycolide copolymers, sucrose acetate isobutyrate (SAIB, i.e. SABER platform), and lactide/caprolactone copolymers.

In certain embodiments, the matrix includes an emulsifier.

In certain embodiments, the emulsifier is sucrose acetate isobutyrate (SAIB).

In certain embodiments, the matrix includes a solvent that allows it to be administered through a small-gauge needle.

In certain embodiments, the matrix is administered via injection or via an in-situ gel upon intramuscular injection for controlled release of API.

In certain embodiments, the controlled release agent includes a prescribed dose of progesterone (P4) and/or luteinizing hormone (LH).

In certain embodiments, the API is a dual hormone treatment.

In certain embodiments, the controlled release LH agent causes immediate regression of the dominant follicle in the animal.

It is an object of the present invention in one formulation where the regression of the dominant follicle begins immediately post administration of the controlled release agent. In another formulation regression of the dominant follicle begins 6-8 days post administration of the controlled release agent.

In certain embodiments, the LH surge causes follicles in the bovine animal to ovulate.

In certain embodiments, the controlled release agent includes two controlled release doses of LH with each LH dose releasing into the bloodstream days apart.

In certain embodiments, the controlled release agent includes only a single dose of LH

In certain embodiments, the LH will indirectly stimulate an endogenous LH surge to culminate in synchronous ovulation.

In certain embodiments, LH will cause DFR.

In certain embodiments, LH is encapsulated and included with P4.

In certain embodiments, the method suppresses estrus in female cattle, sheep, goats, camels and other traditionally mono-ovular species of animals other than humans.

In certain embodiments, the formulation is a suspension or is an extended release powder for suspension.

In certain embodiments, the engineered hormone microsphere matrix comprises at least one polymer.

In certain embodiments, the hormone microsphere matrix comprises poly lactic acid(PLA) or poly lactide-co-glycolide (PLGA).

In certain embodiments, the hormone microsphere matrix is suspended in a buffer.

In certain embodiments, the controlled release agent includes a prescribed dose of progesterone (P4) and/or luteinizing hormone (LH).

In certain embodiments, the controlled release agent includes only a single dose of progesterone (P4).

In certain embodiments, the controlled release agent includes two controlled release doses of LH with each LH dose releasing into the bloodstream days apart.

In certain embodiments, the controlled release agent includes only a single dose of LH

In certain embodiments, the LH will indirectly stimulate an endogenous LH surge to culminate in synchronous ovulation.

In certain embodiments, LH will indirectly cause DFR.

In certain embodiments, LH is encapsulated and included with P4.

In certain embodiments, the formulation replaces 2 GnRH injections with a single LH injection.

Other objects of the invention are achieved by providing a method for multiple ovulation embryo transfer (MOET), OPU, TAI and TET comprising administering a dual hormone formula comprising a prescribed volume of LH and P4 to a bovine animal, wherein the LH in the dual hormone formula is available immediately to the bloodstream of the bovine animal, while the P4 is available to the bloodstream of the bovine animal over a desired period of time via an engineered controlled release.

In certain embodiments, the formula is administered by an injection.

In certain embodiments, the dual hormone formula is for dominant follicle removal.

In certain embodiments, the P4 is released over 7 days after the administration of the dual hormone formula.

In certain embodiments, the P4 is released over 14 days after the administration of the dual hormone formula.

In certain embodiments, the controlled release of P4 is followed by a controlled release LH surge resulting in ovulation.

In certain embodiments, the formula is administered by an injection.

In certain embodiments, the dual hormone formula is for dominant follicle removal.

Other objects of the invention are achieved by providing a biodegradable controlled release (CR) polymer system to a bovine animal.

In certain embodiments, the system includes an intramuscular (IM) injection of controlled release (CR) microparticles.

In certain embodiments, the CR microparticles are molecules of API (P4/LH) encapsulated in engineered thicknesses of poly lactide-co-glycolide (PLGA).

In certain embodiments, as the PGLA layer biodegrades at various engineered times, the API (P4, or P4/LH, or P4/LH) is released into the bloodstream.

In certain embodiments, the CR microparticles are then placed into a “liquid polymer matrix solution.”

In certain embodiments, when the encapsulated CR microparticle solution is injected and contacts aqueous body fluids, it forms a solid (an in-situ gel, a “depot”).

In certain embodiments, the release of the individual API encapsulated particles into the bloodstream occurs over time as the polymer in-situ gel matrix depot biodegrades and the API is released from the depot.

In certain embodiments, non-encapsulated API molecules can be “dissolved” into the liquid polymer matrix and enter circulation over time as the polymer in-situ gel degrades.

In certain embodiments, the engineered microparticles allow the API to be released at desired times.

In certain embodiments the inventive injected controlled release formulation comprises 1.5 g-5.0 g of progesterone (P4) and 25 mg-50 mg of luteinizing (LH) per dose.

In certain embodiments of the inventive injected controlled release formulation, the progesterone (P4) remains in system for 14 days with a burst release of luteinizing (LH) at day 7.

In certain embodiments of the inventive injected controlled release formulation, the progesterone (P4) climbs to 3-4 ng/ml in circulation in approximately 6 hours after injection and sustains an approximate level of 3-4 ng/ml over 13 days, and then drops to approximately <0.5 ng/ml.

In certain embodiments of the inventive injected controlled release formulation progesterone (P4) climbs to 3-4 ng/ml in circulation in approximately 6 hours after injection and sustains an approximate relative flat level of 3-4 ng/ml over 7 days, and then quickly drops to approximately <0.5 ng/ml.

Other objects of the invention and its particular features and advantages will become more apparent from consideration of the following drawings and accompanying detailed description. It should be understood that the detailed description and specific examples, while indicating the preferred embodiment of the invention, are intended for purposes of illustration only and are not intended to limit the scope of the invention.

### DETAILED DESCRIPTION OF THE INVENTION

In the following description, numerous details are set forth for the purpose of explanation. However, one of ordinary skill in the art will realize that the invention may be practiced without the use of these specific details.

The application incorporates by reference the contents of U.S. Pat. No. 5,679,377 entitled “Protein Microspheres and Methods of Using Them.”

In certain embodiments, it is contemplated that the hormone microsphere matrix comprises at least one polymer.

In certain embodiments, it is contemplated that the hormone microsphere matrix comprises poly (lactide-co-glycolide) (PLGA) or poly (lactic acid) (PLA).

In certain embodiments, it is contemplated that the hormone microsphere matrix comprises an organic polymer.

In certain embodiments, it is contemplated that superovulation in the bovine animal involves stimulating at least two primordial follicles in the bovine animal to mature simultaneously.

In certain embodiments, it is contemplated that the controlled release agent includes a single dose of progesterone (P4) and zero to two doses of luteinizing hormone (LH).

In certain embodiments, it is contemplated that the controlled release agent includes a single dose of progesterone (P4) and zero to two doses of LH.

In certain embodiments, it is contemplated that the controlled release agent causes immediate regression of the dominant follicle in the bovine animal via LH.

In certain embodiments, it is contemplated that the regression of the dominant follicle begins immediately post administration of the controlled release agent.

In certain embodiments, it is contemplated that P4 is provided at a constant, prescribed concentration from hours 8 to 390 more/less post administration.

In certain embodiments, it is contemplated that the controlled release agent includes a calculated surge (burst) of LH between hours 0-12, and again between hours 145-182 post administration.

In certain embodiments, it is contemplated that the LH surge causes follicles in the bovine animal to ovulate.

In certain embodiments, it is contemplated that the hormone microsphere matrix is administered via a preferred intra-muscular (IM) injection.

In certain embodiments, it is contemplated that the hormone microsphere matrix is administered via a subcutaneous (SQ) method.

In certain embodiments, it is contemplated that the formulation is a suspension or is an extended release powder for suspension.

In certain embodiments, it is contemplated that the formulation is an in-situ gel.

In certain embodiments, it is contemplated that the hormone microsphere matrix comprises at least one polymer.

In certain embodiments, it is contemplated that the hormone microsphere matrix comprises poly(lactic acid) (PLA) or poly (lactide-co-glycolide) (PGLA).

In certain embodiments, it is contemplated that the hormone microsphere matrix is suspended in a buffer.

In certain embodiments, it is contemplated that the controlled release agent includes a single dose of progesterone (P4) and zero to three doses of luteinizing hormone (LH).

In certain embodiments, the agent is an engineered, sustained release complex.

In certain embodiments, the agent is an extended release powder for suspension, administered as a 10-20 ml IM single injection via 16 ga×1.5″ needle.

In certain embodiments, the PLA or PGLA is the release rate controlling polymer.

In certain embodiments, the formulation is a single injection drug for veterinary use using two different hormones in a single dose to achieve desired, different P4 and LH release profiles.

In certain embodiments, the controlled release method would (a) cause immediate regression of the dominant follicle via LH dose, (b) beginning at 48 hours post injection, provide delivery of P4 to the target organs between hours 48-144, (c) followed by a calculated surge (burst) of LH between hours 145-182 post injection.

In certain embodiments, the formulation is biodegradable.

In certain embodiments, the formulation includes encapsulated microparticles or microspheres, or in-situ gel.

In certain embodiments, the biodegradable polymer provides for the parenteral delivery of a defined concentration and volume of P4 and/or LH throughout a specified time period.

In certain embodiments, the formulation is an intramuscular (IM) or subcutaneous (SQ) injection of controlled-release (CR) microparticles. This option contains prescribed quantities of P4 & LH, encapsulated within a polymer having various thicknesses and/or dissolution rates, thereby allowing prescribed amounts of active pharmaceutical ingredients (API) to become released into the bloodstream at pre-determined, designed times.

In certain embodiments, the formulation is an IM injection resulting in an in-situ gel (i.e. a depot) which features biodegradable polymer CR precipitation technology. Biodegradable injectable in situ gel forming drug delivery consists of biodegradable polymers dissolved in a biocompatible carrier. When the liquid polymer system is aspirated from its vial and injected in the body using standard needles and syringes, it solidifies upon contact with aqueous body fluids to form a solid implant.

In certain embodiments, the formulation accomplishes via 2 hormones, as a single injection, specifically: removal of the dominant follicle by LH, followed by the engineered LH surge resulting in ovulation per a defined time schedule.

In certain embodiments, the formulation accomplishes via 2 hormones, as a single injection, specifically: removal of the dominant follicle indirectly by controlled release LH, followed by superstimulation (or superovulation) by P4, followed by controlled release LH if for superovulation, which indirectly results in ovulation per a defined time schedule.

In certain embodiments, the API (i.e. the P4/LH drug formulation which has not been formulated into CR microparticles) is incorporated into the polymer solution, it becomes entrapped within the polymer matrix as it solidifies. Drug release occurs over time as the polymer biodegrades, resulting in release of the P4/LH drug formulation into the bloodstream of a female animal.

### Example 1-MOET Study Proposal

### Desired Plasma P4 & LH for MOET Protocol

Hours 0-156 (days 0-6.5): P4 enters the blood stream achieving and maintaining a concentration of 2.0-3.0 ng/ml creating negative, suppressive feedback on estrogen production thereby inhibiting onset of estrus.
Hours 48-60: LH becomes available in the blood stream as 25 mg more/less of active hormone effectively targeting and causing ovulation (removal) of the dominant follicle present on one or both ovaries.
Hours 157-168 (conclusion of day-7): P4 decreases to 0.2-0.6 ng/ml

### Example 2 Timed AI or ET Study Proposal

### Desired Plasma P4 & LH (Emulates 7&7 Synch) for Timed AI or ET Protocol

Hours 0-168 (days 0-7): P4 enters the blood stream achieving within 12 hours and maintaining a concentration of 2.0-3.0 ng/ml creating negative, suppressive feedback on estrogen production thereby inhibiting onset of estrus
Hours 156-174 (conclusion of day-7): LH becomes available in the blood stream as 25 mg more/less of active hormone effectively targeting and causing ovulation (removal) of the dominant follicle present on one or both ovaries
Hours 169-312 (days 8-13): P4 decreases to 1.0-1.5 ng per ml
Hours 313-336 (day 14): P4 decreases to 0.2-0.6 ng/ml

In the study proposal as described, multiple test formulations are prepared utilizing PLA or PLGA (or both) polymers. Analytical methods are implemented to characterize the hormones in the microsphere matrix. The formulations are placed on in vitro release and analyzed to verify release rate over a four-day period. An option to produce a second formulation for an LH release between hours 145-182 post injection is also presented.

In certain embodiments, both microsphere formulations may be combined into a single dose to achieve the different P4 and LH release profile.

The scope of the formulation development phase consists of a single round with 8 microsphere formulations of Poly Active polymer which will be varied in polymer composition and water-to-polymer ratio in accordance with the target product profile as described in Table 2. The formulation rounds are at the 50 to 100 scale.

The process is engineered to produce microspheres in the 50-70 micron diameter range. This size range is suitable for delivery through a 20 ga needle.

### Table 2: Target Product Profile

After preparation of the microspheres, a grinding method is applied to instantaneously release some of the encapsulated API. In case the microsphere processing activity caused hormone degradation, this will be assessed by HPLC and SOS-PHAGE analysis of the released hormone.

The microspheres will be further analyzed for morphology, particle size distribution, and API content.

An in vitro release study will be performed, considering the intended duration of release and the burst requirements with maximum 10 time points (for example 1 h, 6 h, 12 h, 24 h, 36 h, 48 h, 96 h). The in vitro samples will be analyzed by HPLC and SOS-PHAGE for activity.

An injectability study will be performed on one of the select prototype formulations. Microspheres shall be resuspended in buffer and drawn through successively smaller needle gauges. This study shall establish the optimal balance between microsphere concentration, injection volume and needle gauge.

### Additional Embodiments

An objective of the instant invention is elimination of inserted progesterone impregnated devices (“PDs”) to control the estrus cycle of bovine for seven to fourteen days regardless of their natural twenty-one-day estrous cycle; and replaced by an injectable two-hormone sustained release drug allowing for the synchronization of many bovine to be artificially inseminated (AI) and/or be implanted with an embryo (ET),

### EXAMPLES

In certain embodiments, the invention involves a new, controlled release injectable (progesterone w/LH) that has CR particles within in-situ gel. Progesterone is frequently referred to as P4. P4 is a steroid hormone, LH is a glycoprotein hormone. The proposed combination shall be referred to as P4+LH.

In certain embodiments, the invention involves controlled release particles within an in-situ gel which provided controlled release of the hormones P4, as well as LH.

P4+LH may be comprised of a defined amount (1.5 g) of CR particles of progesterone (commonly abbreviated as P4) and also a defined amount of CR particles of LH (50 mg if Example-A below, 25 mg in Example-B), all within the in-situ gel.

The function of P4+LH is to control the estrus cycle of cattle for 7 or 14 days (whichever protocol is being followed) thereby allowing the cattle owner to synchronize the estrous cycle of many cattle (regardless of where each animal is in her 21-day cycle) so they all come into heat (into estrus, i.e. heat) on the same day.

Practical example: bring 100 cows or heifers into heat on the same day so they can be (1) artificially inseminated (AI), or (2) so embryo transfer (ET) technicians can transfer a fresh, or a frozen-thawed embryo into each animal.

Note: For the past 25 years groups of cattle have been synchronized by hand-inserting a progesterone-impregnanted device (PD), commercial trade-name CIDR®, into the vagina, then bringing her back into the stall a week later to manually remove the PD (& also inject her with a prostaglandin). One-two days after PD removal, she is brought back (3rd time) into the stall and injected with an LH-releasing hormone to induce ovulation and is inseminated at that time, or receives an embryo a week later.

Replacing CIDR insertion and removal with sustained release P4+LH reduces labor & increases user and animal safety by reducing the number of days that users handle each cow by at least one handling day.

In the most popular 7&7 synch scenario (refer to attached paper) the PD is inserted and remains in the vagina 14 days; 7 days after insertion she is brought into the stall for a GnRH injection, and 7 days later she is again brought into the stall to manually remove the PD and inject her with a prostaglandin.

### Example A

A new, proposed P4+LH formulation to replace the CIDR when used in the popular CIDR 7&7 (14-day) synchronization scenario when inseminating, or transferring embryos into cows or heifers.

Day 1 (& regardless of where she is in her cycle): P4+LH is injected, P4 remains in system for 14 days. Circulating P4 climbs to 3-5 ng/ml in circulation within 6 (more/less) hours of injection, sustains a relatively flat level of 3-5 ng/ml over 14 days, then quickly drops to <0.5 ng/ml by end of day 14. A technician would inject the cow with prostaglandin at day-14.

A burst-release of 25 mg LH occurs at day-7.

A burst-release of 25 mg LH occurs between day 16-17 at/close to time of insemination.

### Example B

A new, proposed formulation to replace the CIDR used in the traditional CIDR 7-day synch scenario for superovulation (MOET) prior to insemination.
Day 1 (& regardless of where donor is in her cycle): P4+LH is injected, where P4 remains in system for 7 days, dropping to <0.5 ng/ml by end of day 7
Day 3: FSH-LH combo is injected which causes superovulation
Day 7: Sustained release P4+LH has dropped to nil and a technician would inject the cow with prostaglandin.
Day 9: A burst-release of 25 mg LH occurs between day 8-9 at/close to time of insemination.

It is contemplated in preferred embodiments of the instant invention the need for the insertion and/or removal of PDs is eliminated; rather; utilizing the inventive P4+LH disclosed method and formulation(s) in protocols designed to induce superstimulation and/or superovulation in bovine, and synchronization of cycles for timed insemination TAI and timed embryo transfer TET.

Having thus described several embodiments for practicing the inventive method, its advantages and objectives can be easily understood. Variations from the description above may and can be made by one skilled in the art without departing from the scope of the invention.

Accordingly, this invention is not to be limited by the embodiments as described, which are given by way of example only and not by way of limitation.

### REFERENCES

Alcivar AA. et al. 1992. Endocrine changes in beef heifers superovulated with follicle stimulating hormone (FSH-P) or human menopausal gonadotropin. J Anim Sci; 70:224-231.
Armstrong DT. 1993. Recent advances in superovulation of cattle. Theriogenology 39:7-24.
Blondin et al. 2002. Manipulation of follicular development to produce developmentally competent bovine oocytes. Biology of Reproduction 66:38-43
Bo, G.A. et al., 1994. Superovulatory response to a single subcutaneous injection of Folltropin-V in beef cattle. Theriogenology, Nov. 1, 1994, Vol. 42, Issue 6, Pages 963-975
Callesen et al. 1986. Preovulatory endocrinology and oocyte maturation in superovulated cattle. Theriogelology 25:71-86.
Cooke et al. 1997. Circulating FSH isoform patterns during recurrent increases in FSH throughout the oestrous cycle of heifers. J. of Reproduction and Fertility 110:339-345.
Driancourt MA. et al. 1992. Effect of superovulation with pFSH or PMSG on growth and maturation of the ovulatory follicles in sheep. Anim Reprod Sci; 27:279-292.
Goff AK, Greve T, Bousquet D, King WA. 1986. Progesterone and luteinizing hormone profiles in heifers used as oocyte donors. Theriogenology; 26:577-586.
Hyttel et al. 1988. Ultrastructure of in-vivo fertilization in superovulated cattle. J. Reproduction and Fertility, 82:1-13.
KIMURA, K. Superovulation with a single administration of FSH in aluminum hydroxide gel: a novel superovulation method for cattle. The Journal of Reproduction and Development, 62 (5), 423-429 (2016).
Landry et al. 2016. Effect of cow age on the in vitro developmental competence of oocytes obtained after FSH stimulation and coasting treatments. Theriogenology 86:1240-1246.
Laster, DB. 1972. Disappearance of and uptake of 1251 FSH in the rat, rabbit, ewe and cow. J. Reproduction and Fertility, 30:407-415.
Martins, CM, et at. 2012. The effect of timing of the induction of ovulation on embryo production in superstimulated lactating Holstein cows undergoing fixed-time artificial insemination. Theriogenology 78 (5): 974-980.
Nivet et al. 2012, FSH withdrawal improves developmental competence of oocytes in the bovine model. Reproduction (Cambridge, England). 143. 165-71. 10.1530/REP-11-0391.
Nivet et al. 2017. Influence of luteinizing hormone support on granulosa cells transcriptome in cattle. Anim Sci J. doi: 10.1111/asj.12856
Price, C.A et al. 1999. Effects of superovulation on endogenous LH secretion in cattle, and consequences for embryo production. Theriogenology, Volume 51, Issue 1, 37-46.
Schams et al, 1977. Some studies of the pregnant mare serum gonadotrophin (PMSG) and on endocrine responses after application for superovulation in cattle. Control of Reproduction in the Cow. Martinus Nijhoff, The Hague, pp 122-142.
Soumano K, Price CA. 1997. Ovarian follicular steroidogenic acute regulatory hormone, low-density lipohormone receptor, and cytochrome P450 side-chain cleavage messenger ribonucleic acids in cattle undergoing superovulation. Biol Reprod; 56:516-522.
Takedomi, T. et al. Superovulation of holstein heifers by a single subcutaneous injection of FSH dissolved in polyvinylpyrrolidon. Theriogenology, Volume 43, Issue 7, Pages 1259-1268
Tríbulo, Andrés et al. Superstimulation of ovarian follicular development in beef cattle with a single intramuscular injection of Folltropin-V. Animal Reproduction Science, Volume 129, Issue 1, Pages 7-13.
Yadav, M.C. et al. 1986. Plasma concentrations of luteinizing hormone and progesterone during superovulation of dairy cows using follicle stimulating hormone or pregnant mare serum gonadotrophin. Theriogenology, Volume 26, Issue 4, 523-540.
Yamamoto, M. et al. Superovulation in the cow with a single intramuscular injection of FSH dissolved in polyvinylpyrrolidon Theriogenology, Volume 41, Issue 3, Pages747-755.

1. A method of inducing superovulation in bovine, ovine, camelid and other female animals, the method comprising the steps of:
administering a protein microsphere matrix having a diameter ranging from 50-70 microns to a bovine animal, the protein microsphere matrix including a controlled release agent,
wherein the step of administering the protein microsphere matrix to the bovine animal is performed via a single administration, and
wherein the protein microsphere matrix induces dominant follicle removal, superovulation and timed ovulation of matured follicle cohort in the bovine animal,
wherein the controlled release agent includes a single dose of progesterone (P4) and zero to two doses of luteinizing hormone (LH),
wherein the LH surge causes follicles in the bovine animal to ovulate.

2. The method of claim 1, wherein the controlled release agent causes regression of the dominant follicle in the bovine animal via LH, wherein the regression of the dominant follicle begins immediately post administration of the controlled release agent.

3. The method of claim 1, wherein P4 is provided at a prescribed rate for hours 48-144 post administration.

4. The method of claim 1, wherein the controlled release agent includes a calculated surge (burst) of LH between hours 145-182 post administration.

5. The method of claim 1, wherein the dose of P4 is 75 IU, and wherein the dose of LH is 50 mg.

6. The method of claim 1, wherein the controlled release agent includes only a single dose of progesterone (P4).

7. The method of claim 1, wherein the controlled release agent comprises 1.4 g of progesterone (P4) and 25 mg of luteinizing (LH) per dose.

8. The method of claim 1, wherein the progesterone (P4) is in system for 14 days and there is a burst release of 25 mg of luteinizing (LH) at day 7.

9. The method of claim 1, wherein the progesterone (P4) climbs to 3-4 ng/ml in circulation in approximately 6 hours after injection and sustains an approximate relative flat level of 3-4 ng/ml over 13 days, and then quickly drops to approximately <0.5 ng/ml.

10. The method of claim 1, wherein the progesterone (P4) climbs to 3-4 ng/ml in circulation in approximately 6 hours after injection and sustains an approximate relative flat level of 3-4 ng/ml over 7 days, and then quickly drops to approximately <0.5 ng/ml.

11. The method of claim 1, wherein the controlled release formulation comprises 1.4 g of progesterone (P4) and 25 mg of luteinizing (LH) per dose.

12. The method of claim 1, wherein the progesterone (P4) is in system for 14 days and there is a burst release of 25 mg of luteinizing (LH) at day 7.

13. The method of claim 1, wherein the progesterone (P4) climbs to 3-4 ng/ml in circulation in approximately 6 hours after injection and sustains an approximate relative flat level of 3-4 ng/ml over 13 days, and then quickly drops to approximately <0.5 ng/ml.

14. The method of claim 1, wherein the progesterone (P4) climbs to 3-4 ng/ml in circulation in approximately 6 hours after injection and sustains an approximate relative flat level of 3-4 ng/ml over 7 days, and then quickly drops to approximately <0.5 ng/ml.

15. The method of claim 1, wherein P4 is provided at a prescribed rate for hours 6-336 post administration,
wherein the controlled release agent includes a calculated surge (burst) of LH between hours 168-192 post administration; or
wherein the controlled release agent includes a calculated surge (burst) of LH between hours 384-408 post administration.

16. The method of claim 1, wherein the dose of P4 ranges from 35 to 75 IU and the dose of LH ranges from 25 to 75 mg.

17. A drug formulation for inducing superovulation in bovine animals, the formulation comprising:
a hormone microsphere matrix having a diameter ranging from 50-70 microns to a bovine animal, the matrix including a controlled release agent,
wherein the formulation is a suspension or is an extended release powder for suspension,
wherein the controlled release agent includes a single dose of progesterone (P4) and zero to two doses of luteinizing hormone (LH).

18. The formulation of claim 21, wherein the dose of P4 is 75 IU and the dose of LH is 50 mg.

19. The formulation of claim 17, wherein the controlled release agent includes only a single dose of progesterone (P4).

20. The formulation of claim 17, wherein the controlled release agent includes only a single dose of gonadotropin releasing hormone (LH).
